(19) United States
(12) Patent Application Publication
(10) Pub. No.: US 2010/0268036 A1
(43) Pub. Date: Oct. 21, 2010

(54) Surgical retraction device

(75) Inventors: Christoph Rothweiler, Donaueschingen (DE); Pedro Morales, Tuttlingen (DE); Markus Nesper, Tuttlingen (DE); Dieter Weisshaupt, Immendingen (DE)

Correspondence Address: Lipsitz & McAllister, LLC, 755 MAIN STREET, MONROE, CT, 06468, US

(73) Assignee: Aesculap AG, Tuttlingen (DE)

(21) Appl. No.: 12/798,608

(22) Filed: Apr. 6, 2010

(30) Foreign Application Priority Data
Apr. 16, 2009 (DE) 10 2009 018 139.3

(51) Int. Cl. A61B 1/32

(52) U.S. Cl. 600/214

(57) ABSTRACT

The invention relates to a surgical retraction device which comprises at least two support arms which are moveable relative to each other and are held directly or indirectly upon one another and upon which at least one surgical restraining element for restraining bones or body tissue is or can be arranged, and which also comprises a support arm positioning mechanism for moving and changing a position of the at least two support arms relative to each other, it is proposed that the support arm positioning mechanism should comprise a coarse adjustment mechanism and at least one fine adjustment mechanism with which a position of the at least two support arms relative to each other can be changed mutually independently.

### CROSS-REFERENCE TO RELATED APPLICATIONS

This application claims the benefit of German Patent Application No. 10 2009 018 139.3 filed on Apr. 16, 2009

The present disclosure relates to the subject matter disclosed in German patent application number 10 2009 018 139.3, which is incorporated herein by reference in its entirety and for all purposes

### FIELD OF THE INVENTION

The present invention relates to surgical retraction devices generally, and more specifically to a surgical retraction device comprising at least two support arms which are moveable relative to each other and are held directly or indirectly upon one another, at least one surgical restraining element for holding back bones or body tissue which is arranged or is adapted to be arranged on said arms, and also comprising a support arm positioning mechanism for moving and changing a position of the at least two support arms relative to each other.

### BACKGROUND OF THE INVENTION

Surgical retraction devices of the type described hereinabove are employed in the field of surgery, for example, in heart and thorax surgery. They are used in the form of so-called self-holding retractors in order to obtain clearly visible access to the organs being treated. Hereby, access to the thorax of a patient is effected either between two of his ribs or via the previously divided sternum. In this case, the retractors must withstand large forces in order to keep the point of access to the site of the operation open. In order to allow the forces required for maintaining free access to the site of the operation to be applied in problem-free manner by the utiliser, an operating surgeon for example, and also so as to enable the retraction devices to accommodate these forces, the retraction devices are equipped with a support arm positioning mechanism for changing the position of the at least two support arms relative to each other, in the sense that at least one of them is moved. In the case of the known support arm positioning mechanisms however, the tactility thereof is usually restricted due to the construction thereof. Therefore, it would be desirable to provide a surgical retraction device which can be manipulated with a greater degree of sensitivity.

### SUMMARY OF THE INVENTION

In accordance with the invention, a surgical retraction device comprises at least two support arms which are moveable relative to each other and are held directly or indirectly upon one another. The surgical retraction device further comprises at least one surgical restraining element for holding back bones or body tissue which is or can be arranged on said arms. Moreover, the surgical retraction device comprises a support arm positioning mechanism for moving and changing a position of the at least two support arms relative to each other. The support arm positioning mechanism comprises a coarse adjustment mechanism and at least one fine adjustment mechanism with which the position of the at least two support arms relative to each other can be changed independently of one another.

BRIEF DESCRIPTION OF THE DRAWING FIGURES

The foregoing summary and the following description may be better understood in conjunction with the drawing figures, of which:

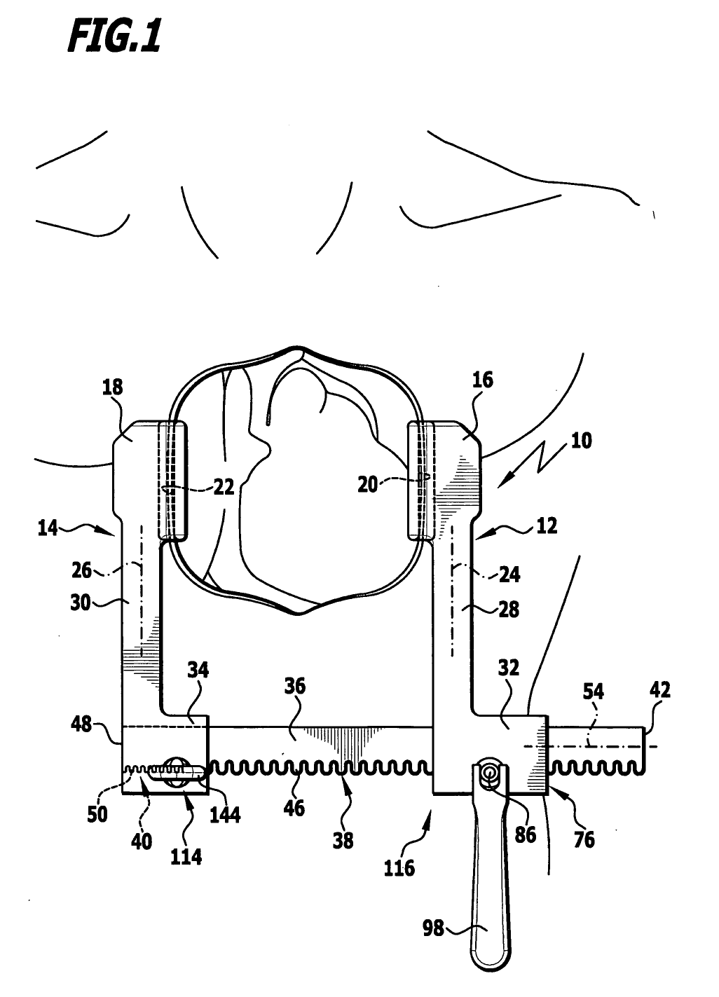

FIG. 1: shows a schematic illustration of a surgical retraction device when spreading a split sternum of a patient;

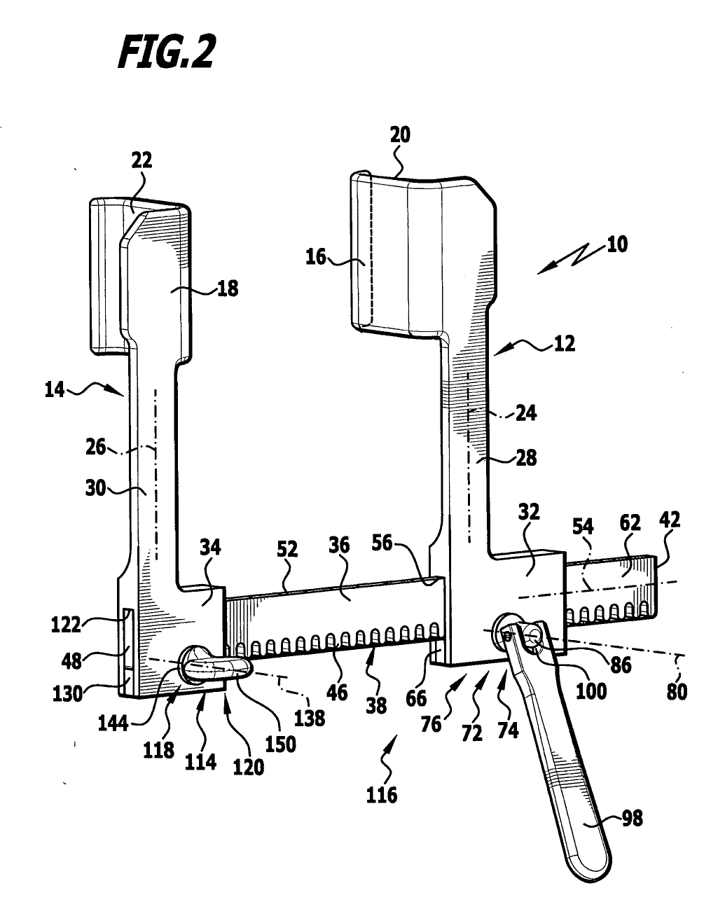

FIG. 2: a perspective view of the retraction device illustrated in FIG. 1;

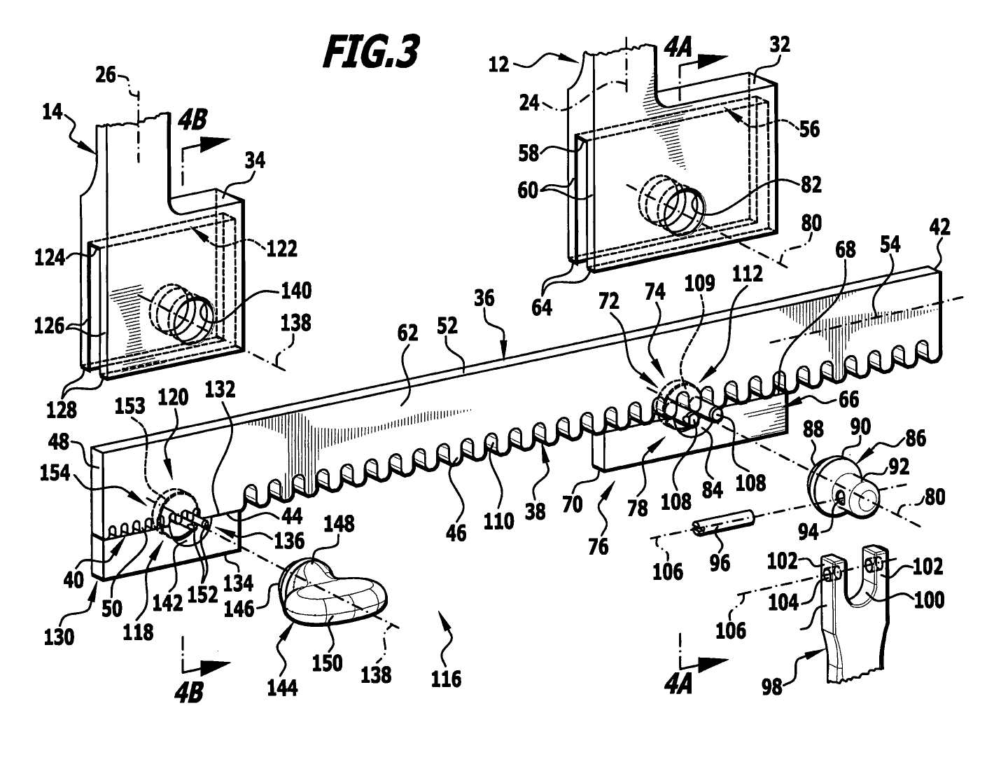

FIG. 3: an exploded illustration of the retraction device depicted in FIG. 2;

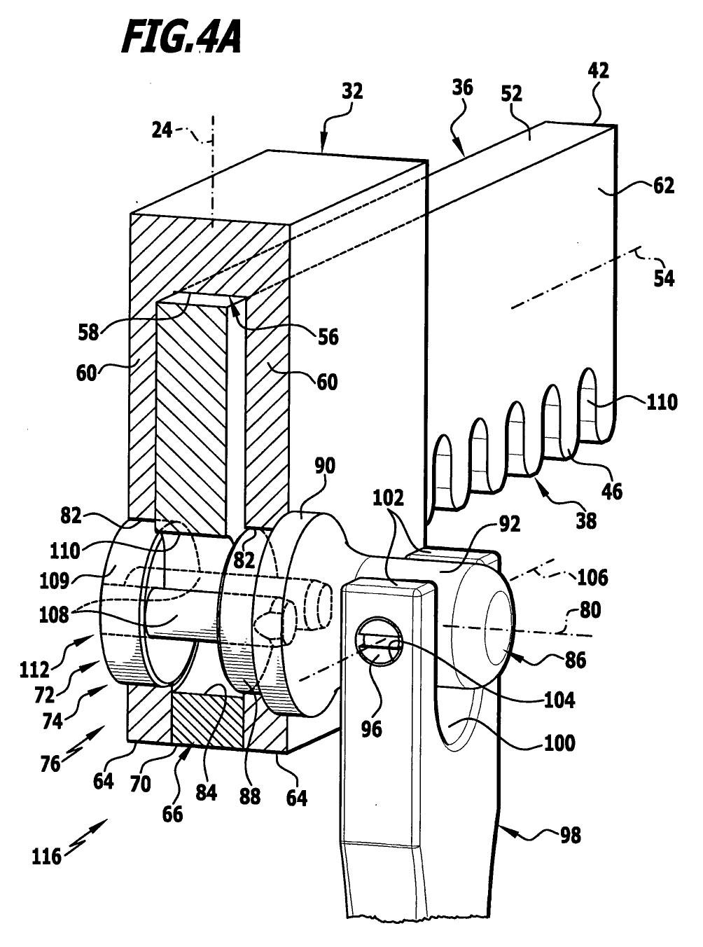

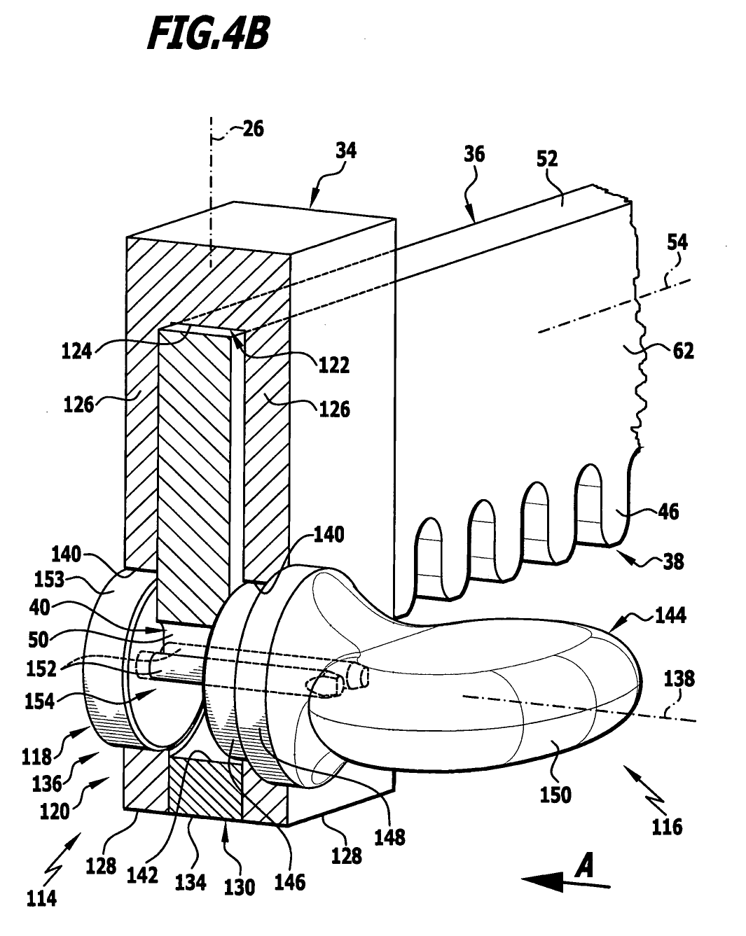

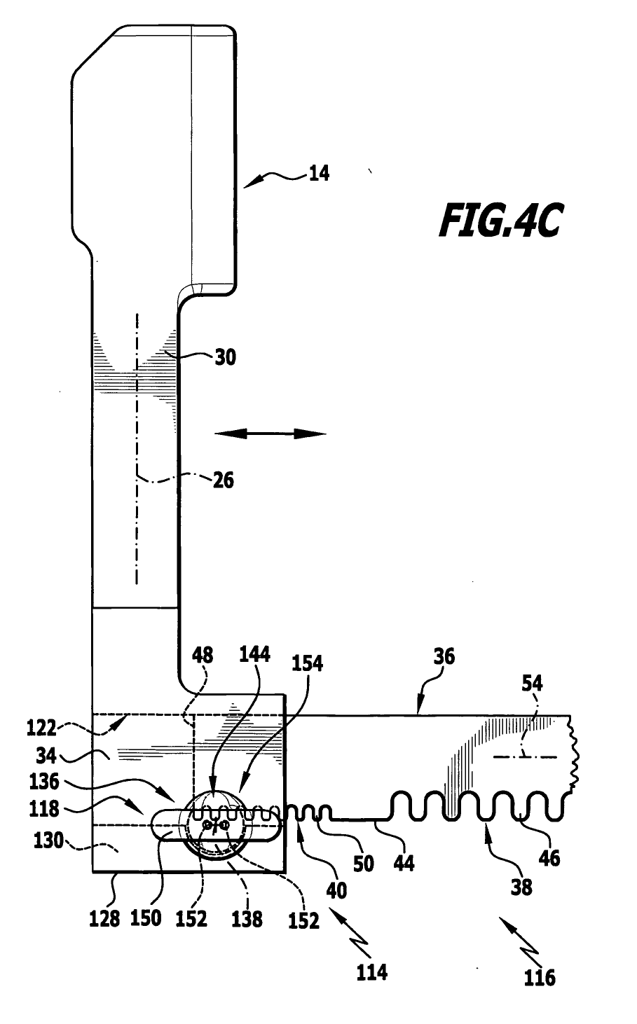

FIG. 4A: a sectional view along the line 4A-4A in FIG. 3;

FIG. 4B: a sectional view along the line 4B-4B in FIG. 3;

FIG. 4C: an enlarged illustration of the end portion of the retraction device depicted in FIG. 2;

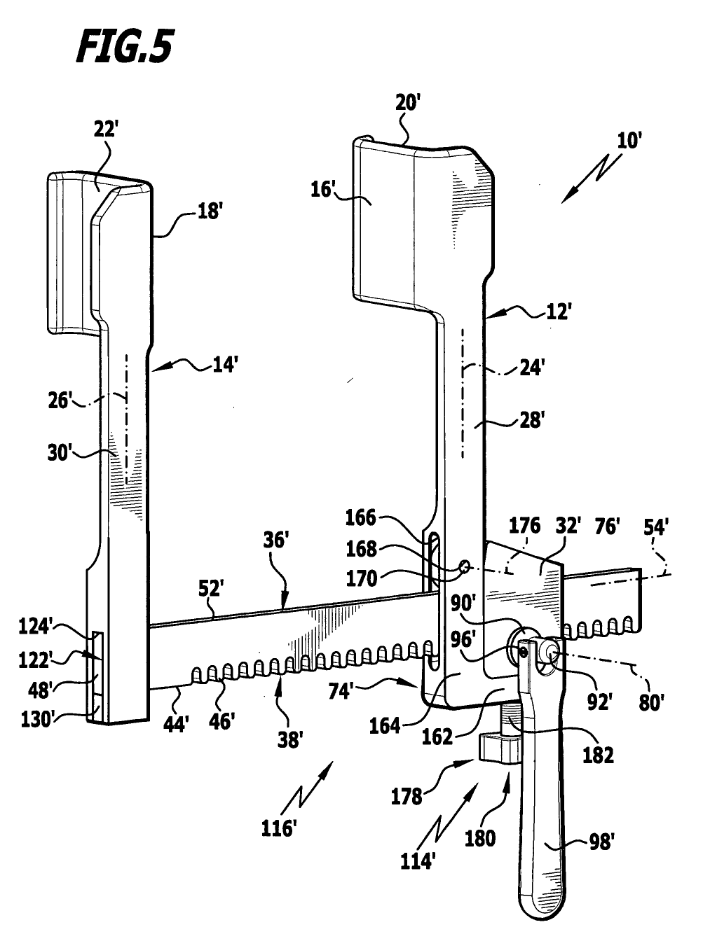

FIG. 5: a perspective view of a further exemplary embodiment of a retraction device incorporating a fixed support arm;

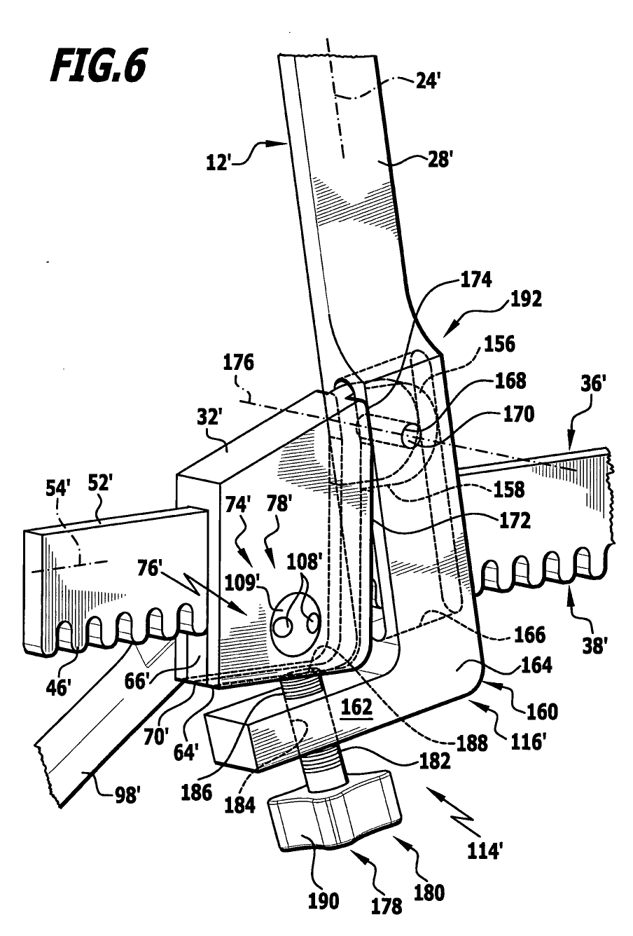

FIG. 6: a further perspective view of the retraction device illustrated in FIG. 5 incorporating a pivoted support arm; and

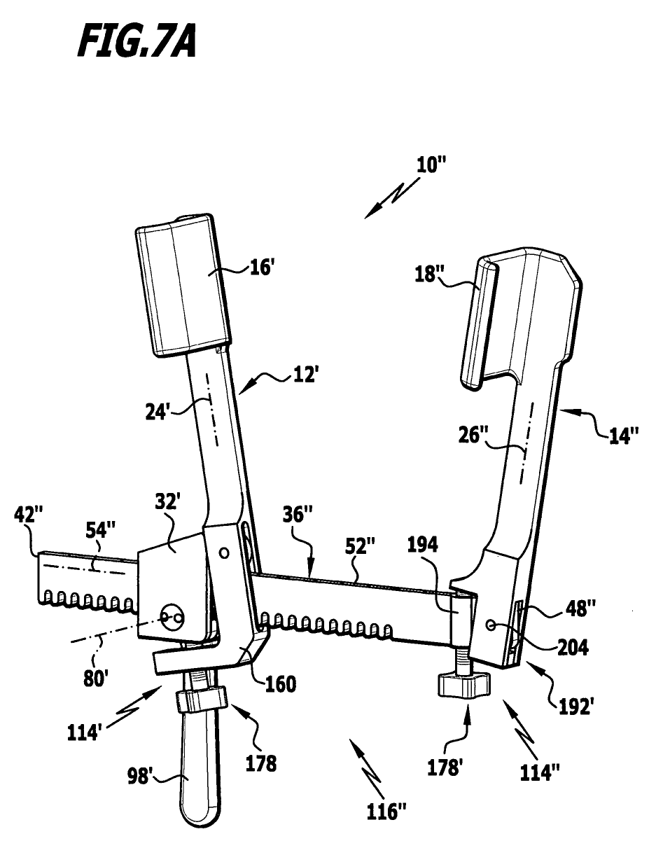

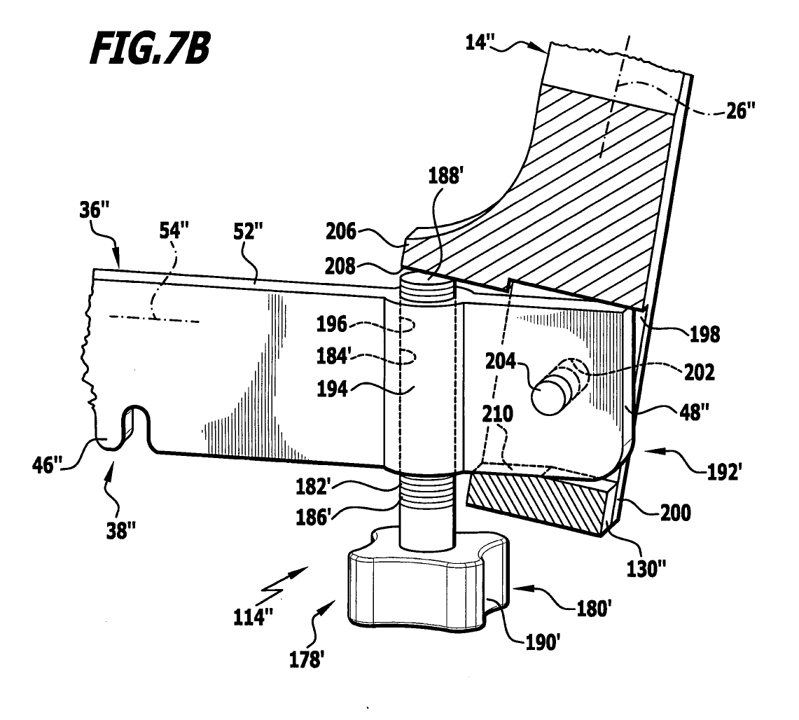

FIG. 7A: a side view of a further exemplary embodiment of a retraction device;

FIG. 7B: a partially sectionalised side view of the front end portion of the retraction device.

### DETAILED DESCRIPTION OF PREFERRED EMBODIMENTS OF THE INVENTION

Although the invention is illustrated and described herein with reference to specific embodiments, the invention is not intended to be limited to the details shown. Rather, various modifications may be made in the details within the scope and range of equivalents of the claims and without departing from the invention.

The present invention relates to a surgical retraction device comprising at least two support arms which are moveable relative to each other and are held directly or indirectly upon one another, at least one surgical restraining element for holding back bones or body tissue which is or can be arranged on said arms, and also comprising a support arm positioning mechanism for moving and changing a position of the at least two support arms relative to each other, wherein the support arm positioning mechanism comprises a coarse adjustment mechanism and at least one fine adjustment mechanism with which the position of the at least two support arms relative to each other can be changed independently of one another.

A support arm positioning mechanism constructed in such a manner enables the spacing between the at least two support arms or a certain positioning thereof to be adjusted with a particularly fine sense of touch. Due to the ability to operate the coarse adjustment mechanism and the at least one fine adjustment mechanism in an independent or separate manner, the process of positioning the at least two support arms, there are frequently just two support arms in such surgical retraction devices, can be effected by pre-positioning them with the coarse adjustment mechanism and then moving them into the desired end positions using the fine adjustment mechanism in a proportioned and sensitive manner. As a result of the relatively coarse support arm positioning mechanisms in conventional arrangements, the feedback or the sense of feeling between the user and the tissues or bones that are being spread is virtually lost so that a cracked sternum and/or fractured ribs can sometimes occur if the utiliser selects the spacing of the at least two support arms relative to one another to be somewhat too great when he is using such a support arm positioning mechanism which cannot be operated with a very fine sense of touch. These problems can no longer occur in the case of the development of the surgical retraction device in accordance with the invention, since, after a coarse positioning of the at least two support arms relative to each other by means of the coarse adjustment mechanism, it is possible to effect a final positioning of the support arms in a highly sensitive manner using the at least one fine adjustment mechanism.

The construction of the surgical retraction device is particularly simple, if the coarse adjustment mechanism and/or the at least one fine adjustment mechanism are constructed such as to move the at least two support arms in parallel with each other towards one another and/or away from one another. In the case of such types of adjustment mechanisms for example, the at least two support arms can be displaced relative to one another.

Moreover, in addition or as an alternative thereto, it can be advantageous if the coarse adjustment mechanism and/or the at least one fine adjustment mechanism are constructed such as to pivot the at least two support arms about at least one pivotal axis towards one another and/or away from one another. For example, the coarse adjustment mechanism can be constructed in the form of a displacing device for the at least two support arms, the at least one fine adjustment mechanism being in the form of a displacing or a pivoting mechanism.

In particular, when the retraction device comprises two support arms, it is expedient for it to also comprise two fine adjustment mechanisms. This permits the position of each support arm to be changed in a sensitive and proportionate manner by the utiliser.

The construction of the surgical retraction device can be simplified in particular, when it comprises a base body on which at least one of the at least two support arms is held in a moveable manner.

It is preferable for two support arms to be held on the base body in moveable manner. However, more support arms could also be held on the base body in moveable manner. The support arms held on the base body in moveable manner can then be moved and positioned individually relative to each other and also with respect to the base body by means of the support arm positioning mechanism.

The surgical retraction device can be manipulated in a particularly simple manner if one support arm is fixed to the base body. In this case, an alteration in the spacing or positioning of the at least two support arms relative to each other is effected by changing the position of a further support arm relative to the base body.

In accordance with a preferred embodiment of the invention, provision may be made for the coarse adjustment mechanism to comprise a coarse drive mechanism for moving one of the at least two support arms and the base body relative to each other. One of the support arms can be roughly positioned relative to the base body or a further support arm with the aid of the coarse adjustment mechanism.

The construction of the surgical retraction device is then particularly simple, if the drive mechanism is constructed in the form of a coarse rack gear drive.

It is expedient, if the coarse rack gear drive comprises a row of coarse teeth on the base body and a coarse bearing element which is mounted on the base body in displaceable manner, if a toothed coarse drive member which is in engagement with the row of coarse teeth is mounted on the coarse bearing element in moveable manner, and if the coarse bearing element is moveable relative to the base body as a result of a movement of the coarse drive member relative to the coarse bearing element. With the aid of a coarse rack gear drive constructed in such a way, a support arm that is held on the coarse bearing element in fixed or moveable manner can be moved relative to the base body in a simple manner.

The construction of the surgical retraction device can be further simplified, if the coarse drive member is constructed in the form of a coarse gear wheel. This is matched, in particular, to the tooth pitch of the row of coarse teeth.

Advantageously, the coarse drive member is mounted on the coarse bearing element such as to be rotatable about a rotational axis of the coarse drive member which runs transversely relative to a longitudinal axis of the base body. In particular, a longitudinal axis of the base body can also be a longitudinal axis of a section of the base body if the base body is not constructed rectilinearly. In each case, the coarse bearing element can be moved in parallel with the longitudinal axis of the base body by the described arrangement by means of a simple rotation of the coarse drive member.

In order to enable the coarse adjustment mechanism to be operated in a simple manner, it is advantageous if it comprises a coarse actuating element for moving the coarse drive member relative to the coarse bearing element.

In accordance with a further preferred embodiment of the invention, provision can advantageously be made for the at least one fine adjustment mechanism to be constructed in the form of a first fine drive mechanism for moving one of the at least two support arms and the base body relative to each other. The first fine drive mechanism enables the at least two support arms to be positioned relative to each other in a sensitive and proportionate manner.

The construction of the surgical retraction device is particularly simple, if the first fine drive mechanism is constructed in the form of a fine rack gear drive.

It is expedient, if the fine rack gear drive comprises a row of fine teeth on the base body and a fine bearing element which is mounted on the base body in displaceable manner, if a toothed fine drive member which is in engagement with the row of fine teeth is mounted on the fine bearing element in moveable manner, and if the fine bearing element is moveable relative to the base body as a result of a movement of the fine drive member relative to the fine bearing element. Expediently, the tooth pitch of the row of fine teeth corresponds to a multiple of the tooth pitch of the row of coarse teeth, for example, to a multiple of between two and five. Putting it another way, this enables the spacing or the relative positioning between the at least two support arms to be adjusted some two to five times more finely.

Advantageously, the fine drive member is constructed in the form of a fine gear wheel.

Preferably the fine drive member is mounted on the fine bearing element such as to be rotatable about a rotational axis of the fine drive member which runs transversely relative to a longitudinal axis of the base body. This arrangement enables the fine bearing element, on which a support arm can be arranged in fixed or moveable manner, to be moved in parallel with the longitudinal axis of the base body by means of a simple rotation of the fine drive member about the rotational axis of the fine drive member.

In order to enable the first fine drive mechanism to be operated in a simple and reliable manner, it is advantageous for the surgical retraction device to comprise a fine actuating element for moving the fine drive member relative to the fine bearing element.

Advantageously, the row of coarse teeth and/or the row of fine teeth respectively extend up to a free end of the base body. This permits the fine bearing element and the coarse bearing element to be separated from the base body and then brought back into engagement therewith in a simple manner. The row of coarse teeth and the row of fine teeth can be formed in the same side face of the base body, although they could also be in different side faces. In particular, it is conceivable for the row of coarse teeth and the row of fine teeth to be formed on the base body in such a manner that they point away in different directions from one another. Furthermore, they could also be arranged or formed on the same section of the base body.

In accordance with a further preferred embodiment of the invention, provision may be made for the at least one fine adjustment mechanism to be constructed in the form of a second fine drive mechanism for the purposes of pivoting a support arm relative to the base body. In particular, the support arm can be arranged on a fine bearing element which can be constructed such as to be fixed or moveable relative to the base body. The second fine drive mechanism makes it possible in a simple way to pivot the at least one support arm relative to the base body in a defined manner with a high degree of sensitivity.

The construction of the surgical retraction device is particularly simple, if the second fine drive mechanism is arranged or formed on the coarse bearing element or on the base body. Self-evidently, it is also conceivable for the second fine drive mechanism to be arranged on a separate bearing element which is held on the base body in moveable manner. As already stated, a bearing element of the second fine drive mechanism could also be arranged or formed such that it is fixed to the base body.

The at least one support arm can be pivoted in a simple manner if the second fine drive mechanism comprises a fine spindle drive. A rotational movement of the fine spindle drive or a part thereof can thus be converted into a pivotal movement of the at least one support arm relative to the base body in a simple manner.

The construction of the fine spindle drive is particularly simple, if it comprises a drive spindle which is in contact with a support arm or the coarse bearing element, and if the support arm is pivotal relative to the base body about a pivotal axis as a result of a movement of the drive spindle. For example, a distal end of the drive spindle can rest on or be applied to the support arm or a projection thereof, be in engagement therewith or coupled thereto, namely, in such a manner that a defined pivotal movement of the support arm relative to the base body is effected as a result of a feeding movement of the drive spindle due to a rotation thereof.

Expediently, the drive spindle can be screwed within a drive spindle guide means. The drive spindle can be rotated about its longitudinal axis and thus implement a screwing movement relative to the drive spindle guide means.

The construction of the surgical retraction device is particularly simple, if the drive spindle guide means is arranged or formed on the base body. Further bearing elements for forming the fine spindle drive are not then necessary.

Preferably the drive spindle guide means is arranged or formed on the coarse bearing element or on the base body. This construction makes it possible to roughly adjust a support arm mounted on the coarse bearing element by means of the coarse adjustment mechanism and then to finely adjust it by means of the second fine drive mechanism which is arranged on the coarse bearing element or on the base body.

In order to change the position of the at least one support relative to the base body in small steps with a fine sense of touch, it can be expedient for the drive spindle to comprise a fine thread.

Furthermore, it is advantageous for the surgical retraction device to comprise a stop mechanism for limiting the movement of the at least two support arms relative to each other. It can thus be ensured that the support arms will not be moved in a completely uncontrolled manner. In particular in the case of heart or thorax surgical treatment, this could have negative consequences for a patient.

In order to ensure that the at least two support arms can remain in a desired position, it is advantageous if the surgical retraction device comprises a locking mechanism for locking the at least two support arms, which are moveable relative to each other in an adjustment position, relative to each other in a retention position. The at least two support arms, the position of which can be changed in the adjustment position, can be fixed relative to each other after the relative positioning desired by the utiliser has been achieved so that the retraction device can then adopt the retention position.

In dependence on the type of surgical treatment that is to be performed, it can be expedient if the support arms are in the form of a straight line or are curved.

A first exemplary embodiment of a surgical retraction device, which is also referred to as a retractor hereinafter, is illustrated in FIGS. 1 to 4 where it bears the general reference symbol 10. It comprises two support arms 12 and 14 which are moveable relative to each other and held indirectly upon one another, and upon which a respective surgical restraining element 16 and 18 is formed in one piece manner. As an option, it would also be conceivable to construct the restraining elements in such a manner that they are connectable to the support arms 12 and 14 in releasable manner. The restraining elements 16 and 18 each have a substantially channel-shaped seating 20 and 22 into which a rib or one half of a split sternum is inserted and can thus be held back by the restraining elements 16 and 18, as illustrated in FIG. 1.

The support arms 12 and 14 define respective longitudinal axes 24 and 26 which run in parallel with one another. In essence, they are defined by a respective flat parallelepipedal support section 28 and 30 which carries the respective restraining element 16 and 18 at one end and a bearing element at the other end thereof, namely, the bearing arm 12 carries a coarse bearing element 32 and the support section 30 a fine bearing element 34. The coarse bearing element 32 and the fine bearing element 34 are both held in moveable manner on a base body 36 of the surgical retraction device 10, whereby the base body 36 is constructed in the form of a parallelepipedal bar or rail.

The base body 36 is provided with two sets of toothing which define a row of coarse teeth 38 and a row of fine teeth 40. Starting from a first end 42, the row of coarse teeth 38 is formed on a narrow side edge 44 of the base body 36 and extends over approximately three-quarters of the overall length of the base body and it comprises perpendicularly projecting rounded teeth 46. Starting from the other end 48 of the base body, the row of fine teeth 40 extends away therefrom and comprises a plurality of teeth 50 which are formed in the same side edge 44. A narrow side edge 52 of the base body 36 extending in parallel with the side edge 44 also extends in parallel with a longitudinal axis 54 defined by the base body 36.

The coarse bearing element 32 is constructed in the form of a right parallelepiped and is provided with a channel-like seating 56 having a width which corresponds to the width of the base body 36. The base 58 of the seating 56 rests on the side edge 52. The side walls 60 and 62 that laterally bound the seating 56 rest against the side faces 62 which connect the side edges 44 and 52 together and the free end faces 64 thereof protrude beyond the side edge 44.

The seating 56 is closed by a parallelepipedal closure body 66, the length of which parallel to the longitudinal axis 54, corresponds to that of the coarse bearing element 32. The upper side 68 of the closure body 66 rests against the teeth 46, the lower side 70 of the closure body 66 is flush with the end faces 64. The coarse bearing element 32 is thus guided on the base body 36 in a defined manner such that it is displaceable in parallel with the longitudinal axis 54.

A coarse drive mechanism 72 serves to provide a defined movement of the coarse bearing element 32 on the base body 36, said coarse drive mechanism being constructed in the form of a coarse rack gear drive 74 and being effective for moving the support arm 12 and the base body 36 relative to each other. The coarse drive mechanism 72 forms a part of a coarse adjustment mechanism 76 with which the position of the support arms 12 and 14 relative to each other can be changed. The coarse rack gear drive 74 comprises the row of coarse teeth 38, the coarse bearing element 32 and a toothed coarse drive member 78 which is in engagement with the row of coarse teeth 38. It is mounted on the coarse bearing element 32 such as to be rotatable about the rotational axis 80 of the coarse drive member which runs transversely relative to the longitudinal axis 54 of the base body 36. The coarse bearing element 32 is provided with a transverse boring 82 which passes through the two side walls 60 and is arranged in such a manner that a recess 84 which is in the form of a hollow semi-cylinder and faces in the direction of the row of coarse teeth 38 is formed in the closure body 66.

A first end of a rotatable body 86 has a flat cylindrical section 88 which is adapted to fit snugly into the transverse boring 82 in one side wall 60 and it does not protrude into the seating 56. Adjoined to the section 88, there is a cylindrical stop body 90 which has an outer diameter that is somewhat larger than the transverse boring 82. An end section 92 adjoining the stop body 90 has a diameter which is somewhat smaller than the diameter of the section 88. In addition, it is provided with a boring 94 which extends transversely relative to the rotational axis 80 of the coarse drive member and serves for seating a bearing pin 96 which protrudes beyond the end section 92 at both ends. It serves for providing a moveable coupling to a coarse actuating element 98 which is constructed in the form of an actuating lever and has a U-shaped end 100 comprising two parallel bearing legs 102 between which the end section 92 is engaged, both said bearing legs being provided with a boring 104 into which the bearing pin 96 is inserted in mutually non-rotatable manner. The outer diameter of the bearing pin 96 is somewhat smaller than the internal diameter of the boring 94 so that the bearing pin 96 and the coarse actuating element 98 affixed thereto are pivotal about a longitudinal axis 106 defined by the boring 94.

Two diametrically opposite gear pins 108 which project from the section 88 and are directed away from the stop body 90 extend in parallel with the rotational axis 80 of the coarse drive member and are spaced by a distance which corresponds to the spacing of the teeth 46 so that the gear pins 108 can engage in two adjacent spaces 110 between teeth. A bearing disk 109 in which the gear pins 108 are secured is inserted as a snug-fit into the transverse boring 82 in the other side wall 60. Upon rotation of the rotatable body 86, the coarse drive element 78, which comprises the section 88 and the gear pins 108 and is constructed in the form of a coarse gear wheel 112, meshes with the row of coarse teeth 38 so that the coarse bearing element 32 and thus the support arm 12 is either moved in the direction of the end 42 when the coarse actuating element 98 is rotated counter-clockwise about the rotational axis 80 of the coarse drive member or is moved in the direction of the end 48 when the rotation is in the clockwise direction.

Furthermore, the retraction device 10 comprises a fine adjustment mechanism 114 which forms a support arm positioning mechanism 116 together with the coarse adjustment mechanism 76. The fine adjustment mechanism 114 is provided for changing the position of the support arm 14 relative to the base body 36. The two support arms 12 and 14 can be moved relative to each other and independently of one another with the aid of the coarse adjustment mechanism 76 and the fine adjustment mechanism 114 whereby the relative position thereof can be changed.

The fine adjustment mechanism 114 is in the form of a first fine drive mechanism 118 which is provided for moving the support arm 14 and the base body 16 relative to each other. It is constructed in the form of a fine rack gear drive 120 which comprises the row of fine teeth 40 and the fine bearing element 34.

The fine bearing element 34 is constructed in the form of a right parallelepiped comprising a channel-like seating 122 having a base 124 which rests on the side edge 52. The seating 122 is bounded laterally by two parallel side walls 126 which rest against the side faces 62 of the base body 36.

The end faces 128 delimiting the free ends of the side walls 126 project beyond the side edge 44. The upper side 132 of a parallelepipedal closure body 130 rests on the side edge 44 and the lower side 134 thereof is flush with the end faces 128. It is inserted into the seating 122 so that the fine bearing element 34 can be displaced in guided manner on the base body 36 in parallel with the longitudinal axis 54.

Furthermore, the fine rack gear drive 120 comprises a fine drive member 136 which is mounted on the fine bearing element 34 such as to be rotatable about the rotational axis 138 of the fine drive member. The axis is defined by a transverse boring 140 which passes through the two side walls 126. Furthermore, the transverse boring 140 forms a recess 142 in the closure body 130 which is open in the direction of the side edge 44 and is in the form of a hollow semi-cylinder. The fine drive member 136 comprises a rotatable body 144 having a flat cylindrical section 146 which engages in snug-fitting manner in the transverse boring 140 in one side wall 126. The section 146 does not protrude into the seating 122. Adjoined thereto is a cylindrical stop body 148, the outer diameter of which is somewhat greater than the diameter of the transverse boring 140. A substantially parallelepipedal fine actuating element 150 is formed on the stop body 148 and it forms a sort of operating knob for the fine drive member 136. The latter furthermore comprises two spaced gear pins 152 which project perpendicularly from the section 146 and extend in parallel with each other from the section 146 and are also parallel with respect to the rotational axis 138 of the fine drive member. The gear pins 152 are arranged at a distance from one another which corresponds to the spacing of the teeth 50. They are arranged diametrically opposite each other taken with reference to the rotational axis 138 of the fine drive member. It is thereby possible for the gear pins 152, which form a fine gear wheel 154 together with the section 156, to be brought into engagement with the teeth 50 and can then mesh therewith. A further bearing disk 153 is inserted in snug-fitting manner into the transverse boring 140 passing through the other side wall 126, the gear pins 152 being additionally secured to said disk. A rotation of the fine actuating element 150 in the clockwise direction moves the fine bearing element 34 in the direction of the end 48, a rotation of the fine actuating element 150 in the counter-clockwise direction leads to a shifting movement of the fine bearing element 34 in the direction of the end 42.

After splitting the sternum, an operating surgeon can insert the retraction device 10 into the cut in such a manner that the seatings 20 and 22 of the restraining elements 16 and 18 each accommodate a part of the split sternum, as illustrated in FIG. 1 for example. By rotating the actuating element 98 in the counter-clockwise direction, the restraining elements 16 and 18 can be moved away from one another. A coarse adjustment using the coarse adjustment mechanism 76 is thus possible.

The spacing of the teeth 50 is about half as great as the spacing of the teeth 46. Consequently, a full revolution of the fine actuating element 150 about the rotational axis 138 of the fine drive member only leads to a feed motion of the fine bearing element 34 which is half of that compared to the feed motion of the coarse bearing element 32 in the course of a full revolution of the coarse actuating element 98 about the rotational axis 80 of the coarse drive member. A surgeon can thereby adjust the spacing between the support arms 12 and 14 and thus between the restraining elements 16 and 18 by means of the fine adjustment mechanism 114 in a significantly more finely proportioned manner than he could using only the coarse adjustment mechanism 76 alone.

A further exemplary embodiment of a surgical retraction device bearing the general reference symbol 10′ is schematically illustrated in FIGS. 5 and 6. Those parts of the retraction device 10′ which are of identical construction or which function in an identical manner to parts of the retraction device 10 are designated hereinafter and in the Figures by means of identical reference symbols bearing an additional dash.

The base body 36′ is constructed in analogous manner to the base body 36, but it only comprises one row of coarse teeth 38′ incorporating teeth 46′. The support arm 14′ is fixed to the end 48′ of the base body 36′ with its longitudinal axis 26′ arranged perpendicular to the longitudinal axis 54′. To this end, a seating 122′ that is formed in analogous manner to the seating 122 is provided at one end of the support arm 14′. A closure body 130′ closes the seating 122′ and abuts the side edge 44′ of the base body 36′, the base 124′ of the seating 122′ abutting on the side edge 52′ of the base body 36′.

The parallelepipedal coarse bearing element 32′ is held on the base body 36′ such that it is displaceable in parallel with the longitudinal axis 54′. It can be moved in analogous manner to the coarse adjustment mechanism 76 by means of the coarse adjustment mechanism 76′.

Other than was the case for the retraction device 10, a fine adjustment mechanism 114′ is not mounted directly on the base body 36′, but rather, on the coarse bearing element 32′. For this purpose, it has a projection which projects from the coarse bearing element 32′ in parallel with the longitudinal axis 54′, the lower side 158 of said projection abutting the side edge 52′. A substantially L-shaped end 160 of the support arm 12′ comprises a leg 162 which sticks out at approximately 90° taken with reference to the longitudinal axis 24′. A section of the L-shaped end 160 extending parallel to the longitudinal axis 24′ is provided with a guide slot 166 having a width which corresponds to the width of the base body 36′ so that the base body 36′ can pass through the guide slot 166. A boring 168 passes through the section 164 in the region of the guide slot 166, said boring also extending through the bearing projection 156. The section 164 is coupled to the bearing projection 156 by means of a bearing pin 170 which is connected to the section 164 in mutually non-rotatable manner and has an outer diameter that is somewhat smaller than the boring 168 passing through the bearing projection 156.

In a basic position of the arrangement, the leg 162 abuts the end faces 64′ of the coarse bearing element 32′, the section 164 abutting a side face 172 of the coarse bearing element 32′ facing in the direction of the end 48′. A stop surface 174 is formed in the region of the bearing projection 156, said stop surface being inclined by about 5° to 15° relative to a plane which is defined by the side face 172 and is oriented perpendicularly to the longitudinal axis 54′. Should the support arm 12′ be swung about a pivotal axis 176 that is defined by the bearing pin 170, then this can only occur until such time as the section 164 strikes against the stop surface 174.

The fine adjustment mechanism 114′ is constructed in the form of a second fine drive mechanism 178 for the purposes of defining the pivotal movement of the support arm 12′ relative to the base body 36′. It is arranged on the coarse bearing element 32′ and comprises a fine spindle drive 180. The fine spindle drive 180 comprises a drive spindle 182 which is provided on the leg 162 in a drive spindle guide means 184 that is in the form of an internally threaded boring. The drive spindle 182 is provided with an external thread in the form of a fine thread 186 which is formed in correspondence with the internal thread of the boring that is formed in the leg 162. A free end 188 of the drive spindle 182 rests on the lower side 70′ of the closure body 66′. At the other end thereof, a rotary knob 190 is formed on the drive spindle 182, this then forming a fine actuating element. The further the drive spindle 182 is screwed into the drive spindle guide means 184 in the clockwise direction, the further the end 188 thereof projects therefrom, whereby the support arm 12′ is pivoted about the pivotal axis 176 until the section 164 strikes against the stop surface 174. Thus, the retraction device 10′ also comprises a stop mechanism 192′ for limiting the movement of the support arm 12′ relative to the support arm 14′ or to the base body 36′. It follows that the drive spindle guide means 184 is formed on the support arm 12′. Alternatively, it could also be arranged or formed on the coarse bearing element 32′. The support arms 12′ and 14′ can be pivoted relative to each other with the aid of the fine adjustment mechanism 114′ for the purpose of continuously increasing the spacing between the restraining elements 16′ and 18′.

A further exemplary embodiment of a retraction device bearing the general reference symbol 10″ is schematically illustrated in FIG. 7. Basically, it differs from the retraction device 10′ only in that the support arm 14″ can be pivoted relative to the base body 36″ by means of a further fine adjustment mechanism 114″. This comprises a second fine drive mechanism 178″ incorporating a fine spindle drive 180′. The fine spindle drive 180′ comprises a drive spindle guide means 184′ which is constructed in the form of a sleeve 194 that is formed on the base body 36″ and is provided with an internal thread 196. It serves for the guidance of a drive spindle 182′ having a fine thread 186′ corresponding to the internal thread 196. At one end of the drive spindle 182′, there is provided a rotary knob 190′ with which the drive spindle 182′ can be screwed into or out of the drive spindle guide means 184′.

The end 48″ engages in a groove 198 in the free end 200 of the support arm 14″. The support arm 14″ is provided with a boring 202 in the region of the groove 198 just as the latter does too, and a bearing pin 204, the longitudinal axis of which is oriented perpendicularly to the longitudinal axis 54″ of the base body 36″, is inserted into said boring. A projection 206 which points in the direction of the end 42″ projects from the support arm 14″ above the side edge 52″. The end 188′ of the drive spindle 182′ is in contact with the projection 206. A stop mechanism 192′ is formed by the base 208 of the groove 198 in conjunction with an upper stop surface 210 of the closure body 130″.

If the drive spindle 182′ is screwed into the drive spindle guide means 184′ in the clockwise direction, the end 188′ presses against the projection 206, whereby the support arm 14″ is swung about a pivotal axis 212, which is defined by the longitudinal axis of the bearing pin 204, until the side face 210 strikes the base 202.

Thus, in the case of the retraction device 10″, there are provided two fine adjustment mechanisms 114′ and 114″ which make it possible to pivot the support arms 14″ and 12′ relative to the base body 36″ independently of one another.

What is claimed is:

1. A surgical retraction device comprising at least two support arms which are moveable relative to each other and are held directly or indirectly upon one another, at least one surgical restraining element for holding back bones or body tissue which is or can be arranged on said arms, and also comprising a support arm positioning mechanism for moving and changing a position of the at least two support arms relative to each other, wherein the support arm positioning mechanism comprises a coarse adjustment mechanism and at least one fine adjustment mechanism with which the position of the at least two support arms relative to each other can be changed independently of one another.

2. A surgical retraction device in accordance with claim 1, wherein at least one of the coarse adjustment mechanism and the at least one fine adjustment mechanism are configured to move the at least two support arms in parallel with each other at least one of towards one another and away from one another.

3. A surgical retraction device in accordance with claim 1, wherein at least one of the coarse adjustment mechanism and the at least one fine adjustment mechanism are configured to pivot the at least two support arms about at least one pivotal axis at least one of towards one another and away from one another.

4. A surgical retraction device in accordance with claim 1, further comprising a base body on which at least one of the at least two support arms is held in a moveable manner.

5. A surgical retraction device in accordance with claim 4, wherein two or more support arms are held on the base body in moveable manner.

6. A surgical retraction device in accordance with claim 4, wherein the coarse adjustment mechanism comprises a coarse drive mechanism for moving one of the at least two support arms and the base body relative to each other.

7. A surgical retraction device in accordance with claim 6, wherein the coarse drive mechanism is constructed in the form of a coarse rack gear drive.

8. A surgical retraction device in accordance with claim 7, wherein the coarse rack gear drive comprises a row of coarse teeth on the base body and a coarse bearing element which is mounted on the base body in displaceable manner, wherein a toothed coarse drive member which is in engagement with the row of coarse teeth is mounted on the coarse bearing element in moveable manner, and wherein the coarse bearing element is moveable relative to the base body as a result of a movement of the coarse drive member relative to the coarse bearing element.

9. A surgical retraction device in accordance with claim 8, wherein the coarse drive member is mounted on the coarse bearing element such as to be rotatable about a rotational axis of the coarse drive member which runs transversely relative to a longitudinal axis of the base body.

10. A surgical retraction device in accordance with claim 4, wherein the at least one fine adjustment mechanism is constructed in the form of a first fine drive mechanism for moving one of the at least two support arms and the base body relative to each other.

11. A surgical retraction device in accordance with claim 10, wherein the first fine drive mechanism is constructed in the form of a fine rack gear drive.

12. A surgical retraction device in accordance with claim 11, wherein the fine rack gear drive comprises a row of fine teeth on the base body and a fine bearing element which is mounted on the base body in displaceable manner, wherein a toothed fine drive member which is in engagement with the row of fine teeth is mounted on the fine bearing element in moveable manner, and wherein the fine bearing element is moveable relative to the base body as a result of a movement of the fine drive member relative to the fine bearing element.

13. A surgical retraction device in accordance with claim 12, wherein the fine drive member is mounted on the fine bearing element such as to be rotatable about a rotational axis of the fine drive member which runs transversely relative to a longitudinal axis of the base body.

14. A surgical retraction device in accordance with claim 12, wherein at least one of the row of coarse teeth and the row of fine teeth respectively extend up to a free end of the base body.

15. A surgical retraction device in accordance with claim 4, wherein the at least one fine adjustment mechanism is constructed in the form of a second fine drive mechanism for the purposes of pivoting a support arm relative to the base body.

16. A surgical retraction device in accordance with claim 15, wherein the second fine drive mechanism is arranged or formed on the coarse bearing element or on the base body.

17. A surgical retraction device in accordance with claim 15, wherein the second fine drive mechanism comprises a fine spindle drive.

18. A surgical retraction device in accordance with claim 17, wherein the fine spindle drive comprises a drive spindle which is in contact with a support arm or the coarse bearing element, and wherein the stop arm is pivotal relative to the base body about a pivotal axis as a result of a movement of the drive spindle.

19. A surgical retraction device in accordance with claim 18, wherein the drive spindle can be screwed within a drive spindle guide means.

20. A surgical retraction device in accordance with claim 19, wherein the drive spindle guide means is arranged or formed on the base body.

21. A surgical retraction device in accordance with claim 19, wherein the drive spindle guide means is arranged or formed on the coarse bearing element or on the support arm.

22. A surgical retraction device in accordance with claim 18, wherein the drive spindle comprises a fine thread.

23. A surgical retraction device in accordance with claim 1, further comprising a stop mechanism for limiting a movement of the at least two support arms relative to each other.

24. A surgical retraction device in accordance with claim 1, further comprising a locking mechanism for locking the at least two support arms, which are moveable relative to each other in an adjustment position, in a retention position relative to each other.

25. A surgical retraction device in accordance with claim 1, wherein the support arms are in the form of a straight line or are curved.
